(19) United States
(12) Patent Application Publication
(10) Pub. No.: US 2026/0335597 A1
(43) Pub. Date: Oct. 29, 2026

(54) MULTI-FRICTION VASCULAR DEVICE

(71) Applicant: Terumo Corporation, Tokyo (JP)

(72) Inventors: Melissa Buzby, Long Beach, CA (US); Anup Dasnurkar, Rancho Santa Margarita, CA (US)

(21) Appl. No.: 19/487,761

(22) PCT Filed: May 24, 2024
(86) PCT No.: PCT/US2024/030999

§ 371 Date: Nov. 25, 2025

Related U.S. Application Data
(60) Provisional application No. 63/468,878, filed on May 25, 2023.

(51) Int. Cl. A61M 25/01; A61M 25/00; A61M 25/09; A61M 29/00

(52) U.S. Cl. CPC A61M 25/0102; A61M 25/0068; A61M 25/09; A61M 29/00; A61M 2025/09133; A61M 2205/0216; A61M 2205/0238; A61M 2207/00

(57) ABSTRACT

Multi-friction dilators may be provided that include a body having an external surface extending from a proximal end to a distal tip and having a first portion, a second portion, and a third portion; wherein: the second portion is disposed between the first portion and the third portion; the external surface in the first portion includes a coating with a first coefficient of friction; the external surface in the second portion exhibits a second coefficient of friction greater than the first coefficient of friction, and the external surface in the third portion exhibits a third coefficient of friction less than the second coefficient of friction.

### CROSS-REFERENCES TO RELATED DISCLOSURES

The present disclosure is a national stage entry of PCT/US 2024/030999 filed on 2024-05-24 and entitled MULTI-FRICTION VASCULAR DEVICE, which claims the benefit of U.S. Provisional Patent Application No.: 63/468,878 entitled “STYLETS AND USES THEREOF” and filed on 2023-05-25, each of which is incorporated herein by reference in its entirety

### FIELD

The present disclosure generally relates to vascular devices. More particularly, the present disclosure relates to vascular devices, such as dilators and stylets, that have multiple regions with different coefficients of friction, including the use and manufacture thereof.

### BACKGROUND

Dilators and stylets are designed to aid in the insertion of a device (e.g., cannula/catheter) into a vascular structure. Dilators and stylets typically are metallic wires or flexible rods inserted into the cavity of the catheter to provide a rigid or semi-rigid structure that eases insertion and positioning of the device. Usually, once the catheter or cannula is introduced into the vasculature, the metal dilator/stylet is removed so only the hollow catheter/cannula remains.

Dilators/stylets have been developed, which are designed to facilitate insertion through resistant valves, and are useful in preventing a catheter/cannula from collapsing during advancement through vasculature. Advancement through vasculature requires a certain flexural rigidity that is high enough to prevent buckling during advancement, yet low enough to avoid trauma to the vasculature during advancement. Another purpose of the dilator/stylet is to fill an open distal end, when present, of a catheter/cannula. In order to manually insert the dilator into a catheter/cannula, and traverse flow valves that may be present in a device assembly that includes the catheter/cannula, the dilator may be coated with a lubricious or hydrophilic coating. However, such a coating reduces manual grip an operator may have on the dilator/stylet, and when combined with the limitations of flexural rigidity on the dilator/stylet (e.g., the requisite flexibility), complicates insertion of the dilator/stylet through any valves present in the catheter/cannula.

Thus, there is a need to be able to both firmly grip the dilator/stylet to advance that device through valves into a catheter/cannula, and at the same time minimize friction from the dilator/stylet that may potentially damage tissue or the catheter/cannula through with the dilator/stylet is advanced.

### SUMMARY

The present disclosure provides a multi-friction dilator, which offers two or more surfaces strategically defined along the longitudinal length of the dilator that are configured to increase gripping or sliding action at certain portions of the dilator. The improved gripping surfaces have, for example, more surface friction than a portion of a surface at the distal tip of the dilators that is configured for improved sliding. The dilators provided herein may be fabricated from a thermoplastic elastomer such as a blend of polyether polymer and polyamide polymer.

The gripping surface may have color and/or tint that are different than the non-gripping surface. This difference facilitates the use of the dilator by a health care professional. The dilators with an improved gripping surface and a different color or tint from the non-gripping surface have several benefits and advantages. Firstly, the improved gripping surface allows for a better grip, facilitating insertion through valves into the catheter, and minimizing the potential for damage to the vasculature. Secondly, the different color or tint of the gripping surface provides an additional visual cue for healthcare professionals during use, helping to prevent confusion and ensure proper use. Additionally, the use of a thermoplastic elastomer, such as a blend of polyether polymer and polyamide polymer, provides flexibility and durability, allowing for multiple uses without compromising the integrity of the dilator. Finally, the implementation of these features does not significantly increase the complexity or cost of production, making the improved dilators an economically viable option.

The present disclosure therefore provides: a dilator for insertion within a catheter, comprising: a body having an external surface extending from a proximal end to a distal tip and having a first portion, a second portion, and a third portion; wherein: the second portion is disposed between the first portion and the third portion; the external surface in the first portion includes a coating with a first coefficient of friction; the external surface in the second portion exhibits a second coefficient of friction greater than the first coefficient of friction, and the external surface in the third portion exhibits a third coefficient of friction less than the second coefficient of friction.

In some aspects of the dilator: the body comprises a thermoplastic elastomer which includes a blend of polyether polymer and polyamide polymer.

In some aspects of the dilator: the thermoplastic elastomer comprises a Shore D hardness of about 20 to about 70.

In some aspects of the dilator: the thermoplastic elastomer comprises a flexural modulus of about 1,000 psi (7 MPa) to about 110,000 psi (760 MPa).

In some aspects of the dilator: the first portion and the third portion are coated with a hydrophilic coating.

In some aspects of the dilator: the first portion and the third portion are coated with a lubricious coating.

In some aspects of the dilator: the distal tip has at least a partially rounded, bulb-shaped end.

In some aspects of the dilator: the first portion extends from the distal tip to a proximal limit at least 1 cm proximal to the distal tip, and the second portion extends proximally at least 1 cm from the proximal limit of the first portion.

In some aspects of the dilator: a lumen is defined in the body that extends axially from a proximal opening at the proximal end, through the body to a distal opening at the distal tip.

In some aspects of the dilator: a lumen is defined in the body that extends axially from a proximal opening at the proximal end, through the body towards the distal tip.

In some aspects of the dilator: a lumen is defined in the body that extends axially from a proximal opening at the proximal end, wherein the lumen includes at least one delivery hole defined in the first portion.

In some aspects of the dilator: a guidewire is disposed within the lumen.

In some aspects of the dilator: the second coefficient of friction is between about 0.05 and about 0.1 and an axial length of the second portion is at least 5 cm.

The present disclosure therefore provides a method of use, comprising: selecting a dilator for insertion into a catheter; gripping the dilator via a gripping surface having a higher coefficient of friction than an average coefficient of friction along a length of the dilator; inserting the dilator through the catheter; in response to reaching a target location in a biological subject accessible via the catheter, withdrawing the dilator from the catheter.

In some aspects of the method of use, the method further comprises: in response to inserting at least a portion of the gripping surface into the catheter, repositioning where the dilator is gripped to a second gripping surface that is located further from a tip of the dilator than the gripping surface.

In some aspects of the method of use, the method further comprises: in response to withdrawing at least a portion of the gripping surface from the catheter, repositioning where the dilator is gripped to a second gripping surface that is located closer to a tip of the dilator than the gripping surface.

In some aspects of the method of use, the method further comprises: in response to reaching the target location, delivering a contrast agent or a therapeutic agent to the target location via delivery holes and a lumen formed in the dilator that are in fluid communication with the target location.

The present disclosure therefore provides a method of manufacture, comprising: receiving a first section and a second section of a base material for construction of a dilator; melding the first section to the second section to form the dilator; covering some, but not all of, the dilator with a sleeve to produce a covered portion and an exposed portion; applying a coating to the dilator; removing the sleeve from the dilator to yield a first portion, corresponding to a first one of the covered portion and the exposed portion, and a second portion, corresponding to a second one of the covered portion and the exposed portion, wherein the first portion and the second portion exhibit different coefficients of friction.

In some aspects of the method of manufacture, the method further comprises: forming, via laser drilling, delivery holes from an external surface of the dilator to a lumen defined axially within the dilator.

In some aspects of the method of use, the coating is applied to the covered portion at a lower concentration than to the exposed portion.

### BRIEF DESCRIPTION OF THE DRAWINGS

The following figures are included to illustrate certain aspects of the present disclosure and should not be viewed as exclusive embodiments. The subject matter disclosed is capable of considerable modifications, alterations, combinations, and equivalents in form and function, as will occur to one having ordinary skill in the art and having the benefit of this disclosure.

FIGS. 1A and 1B show stylets, according to embodiments of the present disclosure.

FIGS. 2A and 2B show dilators, according to embodiments of the present disclosure.

FIGS. 3A and 3B show catheter assemblies that include a dilator, according to embodiments of the present disclosure.

FIG. 4 shows a valve assembly, according to embodiments of the present disclosure.

FIGS. 5A-5F show an insertion sequence of a dilator into a catheter assembly, according to embodiments of the present disclosure.

FIG. 6 is a flowchart of an example method of use for a dilator, according to embodiments of the present disclosure.

FIG. 7 is a flowchart of an example method of fabrication for a dilator, according to embodiments of the present disclosure.

### DETAILED DESCRIPTION

The present disclosure provides a multi-friction dilator, which may also be referred to as a stylet, which offers two or more surfaces strategically defined along the longitudinal length of the dilator that are configured to increase gripping or sliding action at certain portions of the dilator. The improved gripping surfaces have, for example, more surface friction than a portion of a surface at the distal tip of the dilators that is configured for improved sliding. The dilators provided herein may be fabricated from a thermoplastic elastomer such as a blend of polyether polymer and polyamide polymer.

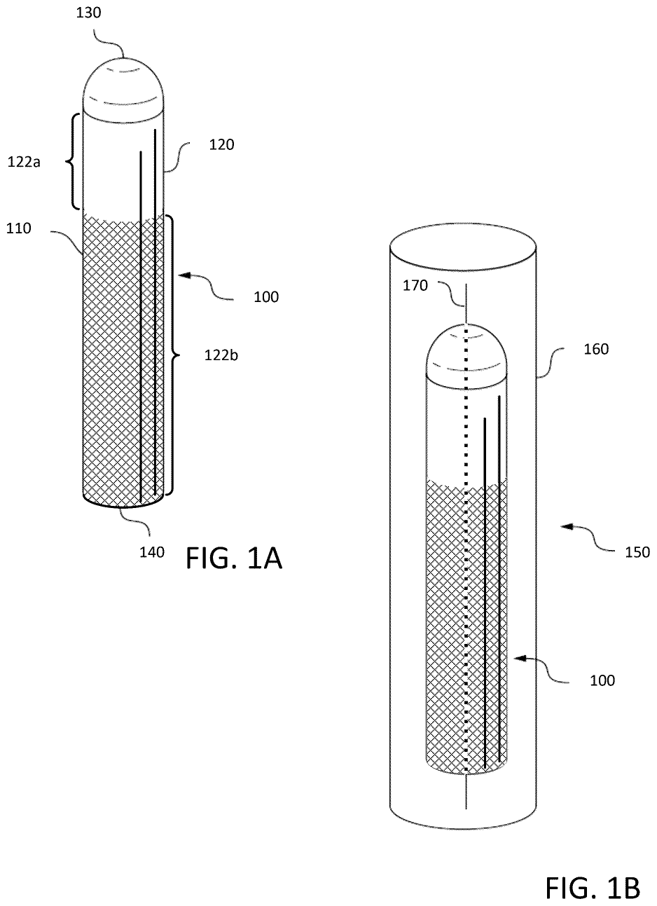

FIGS. 1A and 1B show stylets, according to embodiments of the present disclosure. The illustrated stylet 100 includes a body 110 having an external surface 120, and terminates between a distal tip 130 and a proximal end 140. As shown in FIG. 1B, the stylet 100 may be included in a catheter assembly 150, which includes a cannula/catheter 160 and a guidewire 170 that runs internally to the stylet 100 through respective holes in the distal tip 130 and the proximal end 140.

In various embodiments, the external surface 120 of the stylet 100 may be divided into various portions 122a-b (generally or collectively, portion) that exhibit different surface frictions. The lengths, variations between the different coefficients of friction, hardness (e.g., Shore D hardness), surface coloring, and number of the portions 122 may vary from what is shown in the examples given in FIGS. 1A and 1B, as is discussed in the present disclosure. Additionally, the overall shape and length of the stylet 100 may vary from what is shown in the examples given in FIGS. 1A and 1B.

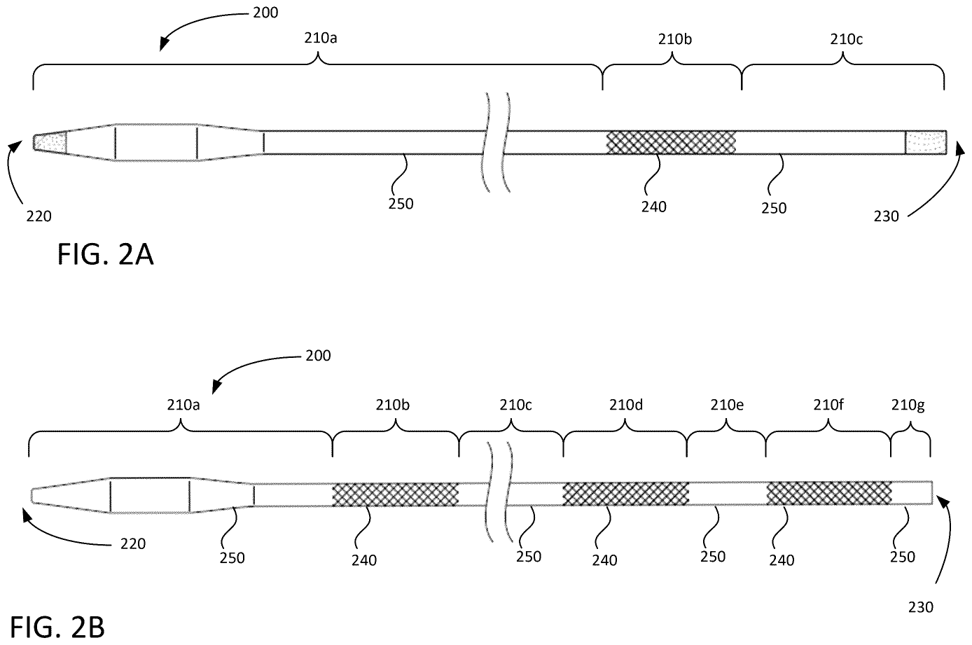

FIGS. 2A and 2B show a dilator 200, according to embodiments of the present disclosure. FIG. 2A shows a dilator 200 has a body that is shown divided into three portions 210a-c (generally or collectively, portions 210), and FIG. 2B shows a dilator that has a body divided into seven portions 210a-g. The portions 210 are arranged between a distal end 220 that is the leading end when inserted through a catheter, and a proximal end 230 that may remain under control of an operator while a length of the dilator 200 is inserted into a biological subject as part of a medical procedure.

The external surfaces of the collective portions 210 exhibit two or more different coefficients of friction in individual portions 210, which are indicated as a gripping surface 240 with a higher coefficient of friction and a lubricious/hydrophilic surface 250 with a lower coefficient of friction, relative to one another. Although FIGS. 2A-2B are illustrated with portions having two different coefficients of friction, in various embodiments, more than or fewer than three/seven portions 210 may be defined on the external surface of the dilator 200, and the various portions may exhibit three or more different coefficients of friction.

For example, the first portion 210a may define a lubricious/hydrophilic surface 250 relative to the second portion 210b, which defines a lubricious/hydrophilic surface 250 relative to the third portion 210c; defining a gradient of decreasing friction in one direction. In another example, the first portion 210a may define a gripping surface 240 relative to the second portion 210b, which defines a gripping surface 240 relative to the third portion 210c; defining a gradient of increasing friction in one direction. In another example, the second portion 210b may define a gripping surface 240 relative to both the first portion 210a and the third portion 210c, but the first portion 210a and the third portion 210c have different coefficients of friction relative to one another. In another example, the second portion 210b may define a lubricious/hydrophilic surface 250 relative to both the first portion 210a and the third portion 210c, but the first portion 210a and the third portion 210c have different coefficients of friction relative to one another. Accordingly, a fabricator may define various coefficients and patterns thereof along a length of the dilator 200.

In various embodiments, these different coefficients of friction are achieved by using different surface coatings in the different portions 210. In some embodiments, at least one portion is coated with a hydrophilic coating. In some embodiments, at least one portion is coated with a lubricious coating.

In some embodiments, the distal end 220 has at least a partially rounded, bulb-shaped end, which allows for easier insertion through a catheter or a blood vessel while guiding a catheter assembly to a target location in a biological subject. In some embodiments, the distal end 220 includes an atraumatic tip.

The lengths of the various portions 210 relative to one another may be configured based on the operation to be performed on the biological subject. In some embodiments, the first portion 210a extends from the distal end 220 to a proximal limit at least 1 centimeter (cm) proximal to the distal tip, and the second portion extends proximally (e.g., from the proximal end of the first portion 210a toward the proximal end 230) at least 1 cm from the proximal limit of the first portion 210a. In some embodiments, the length of the second portion 210b is about 1 cm, about 2 cm, about 3 cm, about 4 cm, about 5 cm, about 6 cm, about 7 cm, about 8 cm, about 9 cm, about 10 cm, or longer.

In some embodiments, the body includes a thermoplastic elastomer which includes a blend of polyether polymer and polyamide polymer. In some embodiments, the thermoplastic elastomer includes a Shore D hardness of about 20 to about 70. In some embodiments, the thermoplastic elastomer includes a Shore D hardness of about 22, about 25, about 27, about 33, about 35, about 40, about 41, about 42, about 46, about 50, about 54, about 58, about 61, about 64, about 66, or about 69.

In some embodiments, the thermoplastic elastomer includes a flexural modulus of about 1,000 pounds per square inch (psi) (7 Megapascals (MPa)) to about 110,000 psi (760 MPa). In some embodiments, the thermoplastic elastomer includes a flexural modulus of about 1,450 psi (10 MPa), about 1,740 psi (12 MPa), about 2,610 psi (18 MPa), about 2,760 psi (19 MPa), about 3,045 psi (21 MPa), about 10,300 psi (71 MPa), about 10,600 psi (73 MPa), about 11,170 psi (77 MPa), about 11,600 psi (80 MPa), about 11,700 psi (81 MPa), about 12,475 psi (86 MPa), about 12,800 psi (88 MPa), about 14,100 psi (97 MPa), about 23,900 psi (165 MPa), about 24,660 psi (170 MPa), about 24,700 psi (170 MPa), about 41,300 psi (285 MPa), about 41,335 psi (285 MPa), about 44,500 psi (307 MPa), about 56,565 psi (390 MPa), about 56,600 psi (390 MPa), about 60,000 psi (414 MPa), about 74,000 psi (510 MPa), about 74,400 psi (513 MPa), or about 102,000 psi (703 MPa).

In some embodiments, the color, tint, or visual pattern of at least one of the portions 210 is different from the other portions 210 so that portions 210 with different coefficients of friction may be distinguished by an operator. In some embodiments, the gripping surfaces 240 are between two lubricous/hydrophilic surfaces 250 (unless the gripping surface 240 is at the distal end 220 or proximal end 230). In some embodiments, each portion 210 is proximal to at least one portion 210 with a different (higher or lower) coefficient of friction. In some embodiments, any two adjacent portions 210 do not have the same coefficient of friction. In some embodiments, the coefficient of friction for a gripping surface 240 is above about 0.05, above about 0.06, above about 0.07, above about 0.08, above about 0.09 or above about 0.1 and the axial length of a portion 210 designated as a including a gripping surface 240 is at least 5 cm in length.

Although illustrated with distinct transitions between adjacent portions 210, in various embodiments, the properties of one portion 210 may blend with the properties of another portion 210 due to manufacturing tolerances or to provide a smooth transition from one portion 210 to another. As used herein, any change from a target property of a given portion 210 to the target value of an adjacent portion 210 that occurs in less than a threshold length along the longitudinal length of the dilator 200 (e.g., 1 millimeter (mm), 2 mm, 3 mm, 4 mm, or 5 mm) may be identified as a distinct transition between the target properties, while any change that exceeds the threshold length may be identified as a gradual transition.

Gradual transitions offer less abrupt changes in various properties than district transitions, and may be beneficial for some properties while in other cases, distinct transitions are more beneficial for other properties. Accordingly, the type of transition between the various target properties in adjacent portions 210 may vary across the properties. For example, a first portion 210a may transition to a second portion 210b via a distinct transition between coefficients of friction, but gradually transition between surface colors.

Similarly, gap transitions may occur between one or more target properties of two adjacent portions 210 due to manufacturing tolerances. For example, when a first coating or other treatment for a first portion 210a does not overlap with a second coating or other treatment for a second portion 210b, leaving an untreated region (e.g., to which neither the first nor second coating or other treatment is applied) between the two portions 210 with properties original to the underlying material, the presence of that untreated region may be identified as a gap transition between the two portions 210. Depending on manufacturing tolerances and design goals for a given target property, these untreated regions may serve as markers between the portions 210 for some properties, be considered benign non-conformances, or lead to rejection of product under quality assurance standards; however, the two portions 210 are still considered to be adjacent for purposes of explanation in the present disclosure.

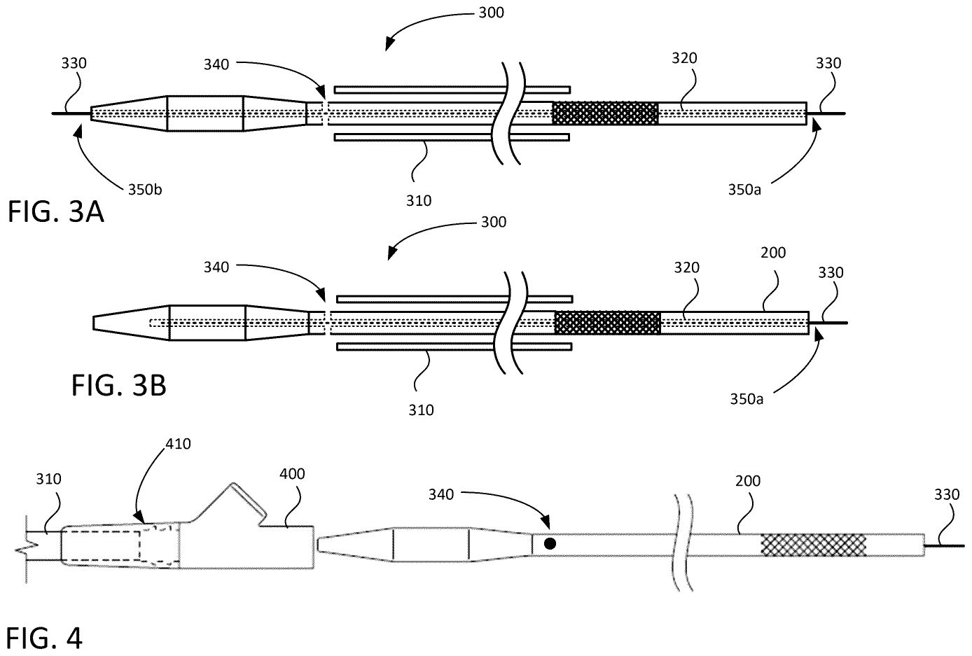

FIGS. 3A and 3B show catheter assemblies 300, according to embodiments of the present disclosure. The catheter assembly 300 include a catheter 310 having an internal lumen 312 in which the dilator 200 is inserted and travels to reach a target location in a biological subject. An internal guidewire 330 is included in the dilator 200 to provide control and additional rigidity to the dilator 200. In various embodiments, the internal guidewire 330 comprises a steel, titanium, or other ductile metal suitable for use in medical procedures.

In some embodiments, the dilator 200 may have a lumen 320 running through the length of the dilator 200 into which the guidewire 330 may be inserted. In some embodiments, as is shown in FIG. 3A, the lumen 320 extends axially between a proximal opening 350a (generally or collectively, opening 350) at the proximal end 230, through the dilatory body, to a distal opening 350b at the distal end 220. In some embodiments, the lumen 320 may omit the distal opening 350b, as is shown in FIG. 3B.

In some embodiments, a portion 210 of the dilator 200, which is located near where the distal end 220 of the dilator 200 or that otherwise emerges from the catheter 310, contains one or more delivery holes 340. These delivery holes 340 may facilitate the delivery of a volume of contrast or therapeutic agent. Accordingly, the delivery holes 340 allow for fluid communication with the target location so that the contrast or therapeutic agent can be flowably delivered via the lumen 320 of the dilator 200 to or near a target location in the biological subject.

In various embodiments, the delivery holes 340 are formed via laser drilling/etching, but may also be formed as voids during an additive manufacturing process, physical drilling, chemical etching, or combinations thereof. These delivery holes 340 are located in a first portion 210a or a second portion 210b in various embodiments. In various embodiments the distal opening 350b may be included as a delivery hole 340. In various embodiments, one, two, three, or more delivery holes 340 are included in the dilator 200. In various embodiments, the one, two, three, or more delivery holes 340 permit flowable delivery of a volume of contrast or therapeutic agent from lumen 320 through distal end 220.

In some embodiments, the overall inner diameter (ID) of the lumen 320 of the dilator 200 (including tapered and straight portions) is large enough to permit the delivery of contrast using a power injector of a predefined pressure without damaging the dilator 200 to achieve a desired flow rate therethrough. In some embodiments, the injector for delivery of contrast may be a manual syringe injector, a dual head injector or a programmable injector.

In some embodiments, the overall inner diameter (ID) of the dilator may be large enough to accommodate a removable internal guidewire 330 with a diameter of about 0.014 inches, about 0.016 inches, 0.018 inches, about 0.020 inches, about 0.022 inches, about 0.024 inches, about 0.026 inches, about 0.028 inches, about 0.030 inches, about 0.032 inches, about 0.034 inches, about 0.036 inches, or about 0.038 inches.

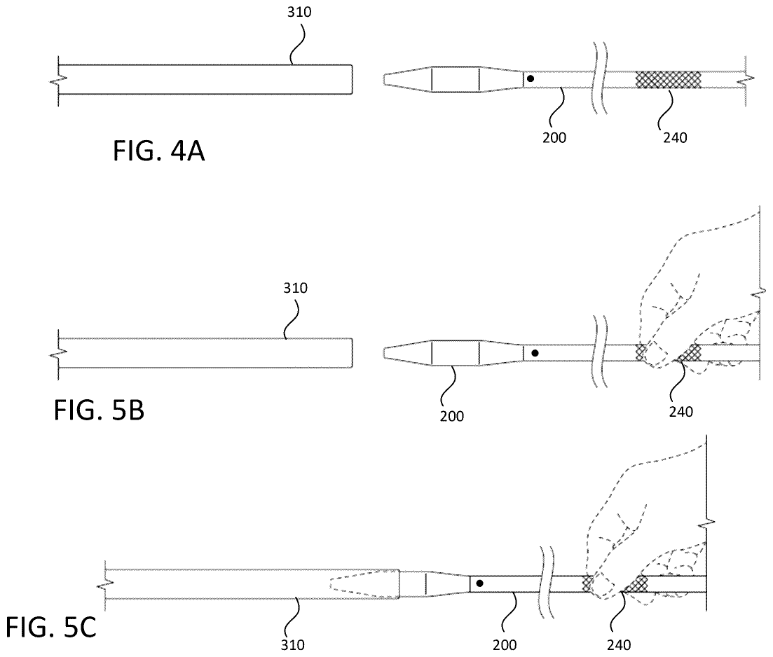

FIG. 4 shows a valve assembly 400, according to embodiments of the present disclosure. The valve assembly 400 includes a valve 410, such as a flow valve or hemostatic valve that the dilator 200 is to traverse when being inserted into or retracted from the catheter 310. By including the portions 210 with different coefficients of friction with lengths that correspond to a distance from a first opening 420a (generally or collectively, opening 420) to a second opening 420b of the valve assembly 400, the operator is provided with a gripping surface 240 outside of the valve assembly 400 by which to grip the dilator 200 for manual or machine-aided insertion, while still having other portions 210 with lower coefficients of friction to slide easily along the catheter 310, the vasculature of a biological subject, etc.

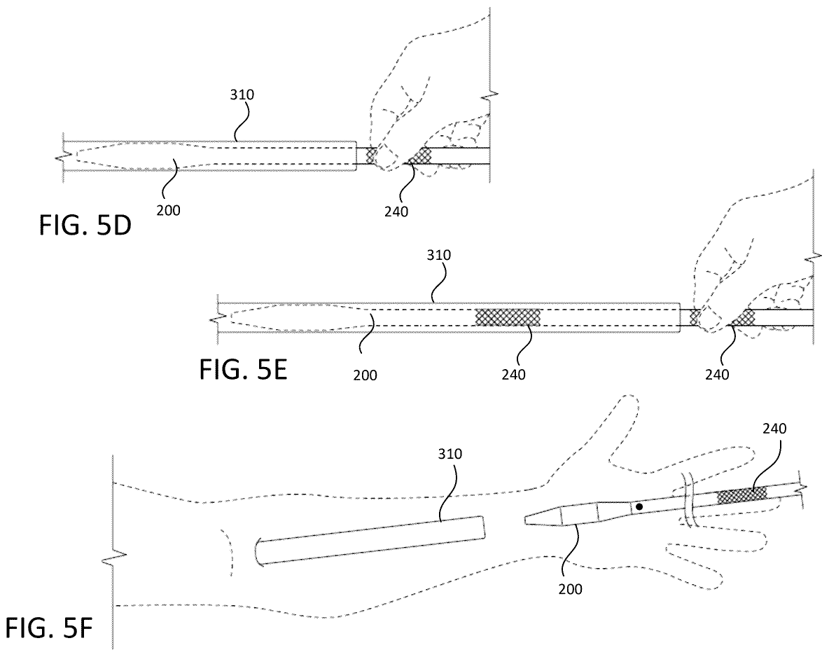

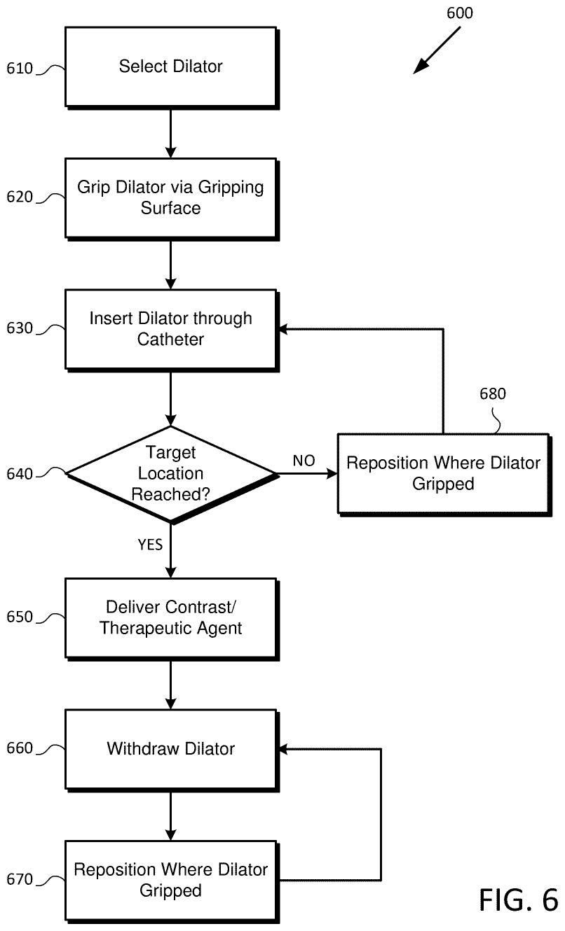

FIGS. 5A-5F show an insertion sequence of a dilator 200 into a catheter assembly 300, according to embodiments of the present disclosure. FIGS. 5A-5F may be understood in conjunction with FIG. 6, which is a flowchart of an example method 600 of use for a dilator, according to embodiments of the present disclosure. FIG. 5F may represent an initial or a final state (e.g., pre-insertion or post-withdrawal) in which a catheter 310 is inserted in a biological subject to provide access to a target location in the biological subject for the dilator 200 (e.g., for thrombectomy, therapeutic agent delivery, etc.).

At block 610, an operator selects a dilator 200 for use with a catheter 310, as is shown in FIG. 5A. In some embodiments, provided herein are kits that include two dilators 200 as described herein. The two dilators 200 may be different or the same. In some embodiments, the two dilators 200 are the same except that the thermoplastic polymer used in each dilator 200 is different.

At block 620, an operator grips the dilator 200 via one or more of the gripping surfaces 240, as is shown in FIG. 5B. The gripping surface 240 provides a location that the operator can securely grip the dilator 200 with reduced effect on the overall friction of the dilator 200 on the interior walls of the catheter 310 or other vessel or tissue in the biological subject. Accordingly, the gripping surface 240 provides a localized region having a higher coefficient of friction that the dilator 200 exhibits on average along the longitudinal length thereof. In some embodiments, the average coefficient of friction is within 90%, 80%, or 70% of the coefficient of friction of the lubricious/hydrophilic surface 250. In some embodiments, the average coefficient of friction is measured across the entire length of the dilator 200, while in other embodiments the average coefficient of friction is measured across the length of the dilator 200 that is inserted into the catheter 310, and as such, the average inserted coefficient of friction may be equal to the coefficient of friction for the lubricious/hydrophilic surface 250 for some or all of the operation and may vary as different numbers of portions 210 with gripping surfaces 240 are inserted or withdrawn.

At block 630, an operator inserts the dilator 200 into the catheter 310 to advance the dilator 200 to a target location in a biological subject accessible via the catheter 310, as is shown in sequence when considering FIG. 5C and FIG. 5D.

In various embodiments, a guidewire 330 may be inserted through the catheter 310 to the target location before the dilator 200 is inserted, in which case, the operator may thread the dilator 200 onto the guidewire 330 as part of block 630. Once the dilator 200 is threaded onto the guidewire 330, the operator may grip a proximal end of the guidewire 330 to ensure that the distal end of the guidewire 330 remains at the target location in the biological subject as the dilator 200 advances along the guidewire 330. As will be appreciated, unless two or more operators are present and coordinating with one another, griping the guidewire 330 by hand leaves the operator with one hand to grip and manipulate the dilator 200.

At block 640, an operator determines whether the dilator 200 has reached a target location. When the dilator has reached the target location, method 600 continues to block 650 (optionally), block 660, and block 670 (optionally). When the dilator 200 has not yet reached the target location, method 600 continues to block 680.

At block 650, the operator optionally delivers a contrast or therapeutic agent through the lumen of the dilator 200 to or near the target site. In embodiments in which one or more delivery holes 340 are defined in the dilator 200, an operator may use the lumen 320 as a fluid pathway, and inject a contrast or therapeutic agent via a manual syringe injector, a dual head injector, a programmable injector, or the like. In various embodiments in which a guidewire 330 was used to aid the dilator 200 in reaching the target location, the guidewire 330 may be removed prior to delivering the contrast or therapeutic agent.

At block 660, the operator withdraws the dilator 200 from the catheter 310, as is shown in sequence when considering FIG. 5D and FIG. 5C. In various embodiments in which a guidewire 330 was used to aid the dilator 200 in reaching the target location, the guidewire 330 may be removed prior to removing the dilator 200, or may be removed after removing the dilator 200.

At block 670, the operator (if needed) repositions where the dilator 200 is gripped for continued withdrawal of the dilator 200 from the catheter 310. For example, the operator may switch from gripping a fourth portion 210d (of a proximally located gripping surface 240) to gripping a second portion 210b (of a tipwardly-located gripping surface 240) to continue having an easy-to-grip portion 210 to hold when the second portion 210b has emerged from the catheter, as is shown in FIG. 5E. Method 600 may then return to block 660 until the dilator 200 is fully withdrawn.

At block 680, the operator repositions where the dilator 200 is gripped for continued insertion into the catheter. For example, the operator may switch from gripping a second portion 210b (of a tipwardly-located gripping surface 240) to gripping a fourth portion 210d (of a proximally-located gripping surface 240) to continue having an easy-to-grip portion 210 to hold while the second portion 210b is about to be or is currently inside the catheter. Method 600 may then return to block 630 for continued insertion towards the target location.

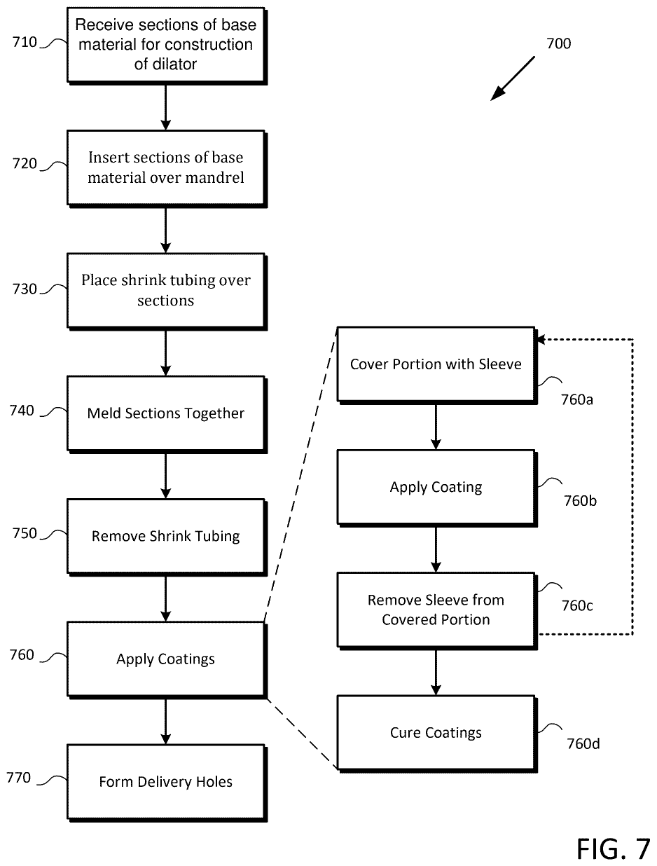

FIG. 7 is a flowchart of an example method 700 of fabrication for a dilator, according to embodiments of the present disclosure. Method 700 begins at block 710, where a fabricator receives extruded sections of the base material for construction of the dilator 200.

In various embodiments, the base material may be various thermoplastic elastomers, such as PEBAX® (available from Arkema France Corp. of Colombes FR) or other thermoplastic elastomers made of flexible polyether and rigid polyamide. For example, the base material may include polyether polymer and polyamide polymer. In various embodiments, the sections may be of the same or different lengths, and may include various pre-defined features that are etched, milled, or otherwise defined in the base material, or may be casted, printed, molded, or otherwise shaped in the fabrication of the individual section. PEBAX® elastomers are block copolymers made up of rigid polyamide blocks and soft polyether blocks. Manipulating these blocks and their relative ratio allows for the creation of a large range of physical properties that span the flexibility spectrum from very hard and rigid to very soft and flexible, without the need for plasticizers. These polymers maintain the highly desirable combination of the toughness traditionally associated with polyamides and the flexibility/elasticity more often seen with polyethers/polyesters. Other examples of thermoplastic elastomers may include but are not limited to thermoplastic urethanes and polyether block amides (PEBA); and polyamides (also known as nylons) and polyamide block copolymers.

At block 720, the fabricator inserts the sections of the base material over a mandrel, with the edges of each section touching an edge of at least one other section.

At block 730, the fabricator places a length of shrink tubing over some or all of the length of the sections of base material, and at least over the edge between two touching edges of adjacently positioned sections. In various embodiments, the shrink tubing may include various commercially available shrink tubing materials suitable for use in medical devices, as will be known to the person of ordinary skill in the relevant art.

At block 740, the fabricator melds the sections together to secure two or more sections of the base material together. In various embodiments, melding may be performed via a heat weld (e.g., melting ends of one or both sections), an adhesive, or a chemical weld.

At block 750, the fabricator removes the shrink tubing from the now-joined sections.

At block 760, the fabricator applies one or more coatings to various portions of the length of the now-joined sections and cures the coating to produce a dilator, according to embodiments of the present disclosure. Different coatings may be applied (or not applied) to various portions of the now-joined sections to impart different coefficients of friction in the different sections At block 760a, the fabricator covers a portion of the dilator that is indicated to not be coated (or to be coated with a lower concentration) with a given coating with a sleeve. In various embodiments, the sleeve may be semi-permeable or include holes (e.g., due to a weave or physical definition) that allows for a controlled lower concentration to be applied to a covered portion. Accordingly, a first portion of the dilator remains exposed, while a second portion is fully or partially covered.

At block 760b, the fabricator applies the given coating to the exposed portions. Depending on the coating, the fabricator may dip, rinse, spray, or use other application tools to apply the given coating to the dilator. Accordingly, the given coating is applied to the exposed portions and not to the covered portions or is applied at a lower rate/concentration than to the exposed portions.

At block 760c, the fabricator removes the sleeve from the dilator, re-exposing the previously covered portions and thereby yielding portions that exhibit different surface properties. In various embodiments, block 760c may return to block 760a for the fabricator to selectively apply different coatings to one or more portions of the dilator (e.g., covering previously exposed portions, applying a different coating to previously covered portions, applying different coats or the same or different coatings, etc.), or may conclude with the currently applied coatings.

At block 760d, once the fabricator has achieved a desired level for the coatings over the length of the dilator, the fabricator cures the coatings (e.g., plasma treats the dilator to impart a higher coefficient of friction to that portion). Block 760d may be performed once for each applied coating, or once as a final sub-operation of block 760 once all desired coatings have been applied, or in multiple sub-operations after some but not all of the desired coatings have been applied.

At block 770, the fabricator defines one or more delivery holes in the dilator, which may be formed via laser drilling. In some embodiments, additionally or alternatively to laser drilling, the fabricator may form the delivery holes via voids present from an additive manufacturing process, physical drilling, chemical etching, or combinations thereof.

Examples of hydrophilic coatings as may be applied by the fabricator may include but are not limited to a combination of UV cure hydrophilic coatings, an aliphatic polyisocyanate resin based on. hexamethylene diisocyanate (HDI) and dissolved in n-butyl acetate and xylene (1:1), propylene glycol methyl ether acetate and a crosslinker mixture. U.S. 20130323291, incorporated herein by reference, discloses hydrophilic and non-thrombogenic polymer for coating of medical devices.

The crosslinker mixture may include but is not limited to one or more of the following compounds: a vinyl compound, ethylene glycol dimethacrylate, an acrylate compound, an epoxy compound, a urethane compound, an isocyanate compound, a triacrylate, a methacrylate, a methacrylic acid, and a hydroxyethyl methacrylate.

The vinyl compound may be one or more of the following compounds: a polyvinyl ethylene glycol, a polyvinyl siloxane, a vinyl pyrrolidone, a vinyl silane, a vinyl polyethylene oxide, and a vinyl polypropylene oxide.

The process described herein may provide one or more of the following advantages: i) consistency: The use of extruded sections and a mandrel may help ensure consistent diameter and shape of the dilators; ii) strength: Melding the sections together may create a strong bond between the sections, providing the dilator with increased strength and durability; iii) customization: The technique may be easily adapted to create dilators with different shapes, sizes, and through-hole configurations to suit different medical applications; iv) efficiency: The use of shrink tubing to hold the sections in place during melding may reduce the need for additional clamping or holding devices, saving time and resources; v) compatibility: The use of thermoplastic elastomers may make the dilators compatible with a wide range of medical uses, procedures and materials; or vi) coatability: The operation of applying a coating over some or all of the dilator may provide additional properties such as lubricity, antimicrobial or surface properties.

When selecting a proper length for the dilator, it is important to consider factors such as the anatomy of the biological subject, the specific medical procedure being performed, and the properties of the device material. Choosing an appropriate length can help avoid issues such as excess friction, discomfort, and tissue damage. If the dilator is too short, the dilator may not be able to reach the desired location in the body of the biological subject, requiring additional insertion attempts and potentially causing trauma to surrounding tissue. In contrast, if the dilator is too long, the dilator may create excess friction and cause discomfort or injury to the biological subject. The length of the dilator should be selected based on the specific anatomical features of the biological and the length required to reach the desired location. In addition, the properties of the device material, such as its flexibility and lubricity, should also be considered when selecting the appropriate length. Furthermore, it is important to consider the specific medical procedure being performed, as certain procedures may require different lengths of dilators. For example, a procedure that involves accessing the urinary tract may require a longer dilator than a procedure that involves accessing the nasal cavity. In sum, selecting the proper length for a dilator is a critical consideration to avoid excess friction, discomfort, and tissue damage. Factors such as the anatomy of the biological subject, the specific medical procedure, and the device material should be taken into account when making these decisions.

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of the disclosure.

Groupings of alternative elements or embodiments of the disclosure disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this disclosure are described herein, including the best mode known to the inventors for carrying out the disclosure. Of course, variations on these described embodiments will become apparent to those of ordinary skill in the art upon reading the foregoing description. The inventor expects skilled artisans to employ such variations as appropriate, and the inventors intend for the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the disclosure so claimed are inherently or expressly described and enabled herein.

Furthermore, references to patents and printed publications may have been made in this specification. Each of the above-cited references and printed publications are individually incorporated herein by reference in their entirety.

As used herein, various chemical compounds are referred to by associated element abbreviations set by the International Union of Pure and Applied Chemistry (IUPAC), which one of ordinary skill in the relevant art will be familiar with. Similarly, various units of measure may be used herein, which are referred to by associated short forms as set by the International System of Units (SI), which one of ordinary skill in the relevant art will be familiar with.

As used herein, various terms provided with reference to the body of a biological subject are to be understood with reference to the standard anatomical position of that biological subject using anatomical terms of location e.g., as set by the International Federation of Associations of Anatomists or the World Associate of Veterinary Anatomists that will be understood by the person on ordinary skill in the relevant art without further explanation.

As used herein, “about,” “approximately” and “substantially” are understood to refer to numbers in a range of the referenced number, for example the range of −10% to +10% of the referenced number, preferably −5% to +5% of the referenced number, more preferably −1% to +1% of the referenced number, most preferably −0.1% to +0.1% of the referenced number.

Furthermore, all numerical ranges herein should be understood to include all integers, whole numbers, or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used in the present disclosure, a phrase referring to “at least one of” a list of items refers to any set of those items, including sets with a single member, and every potential combination thereof. For example, when referencing “at least one of A, B, or C” or “at least one of A, B, and C”, the phrase is intended to cover the sets of: A, B, C, A-B, B-C, A-C, and A-B-C, where the sets may include one or multiple instances of a given member (e.g., A-A, A-A-A, A-A-B, A-A-B-B-C-C-C, etc.) and any ordering thereof. For avoidance of doubt, the phrase “at least one of A, B, and C” shall not be interpreted to mean “at least one of A, at least one of B, and at least one of C”.

It is to be understood that the embodiments of the disclosure disclosed herein are illustrative of the principles of the present disclosure. Other modifications that may be employed are within the scope of the disclosure. Thus, by way of example, but not of limitation, alternative configurations of the present disclosure may be utilized in accordance with the teachings herein. Accordingly, the present disclosure is not limited to that precisely as shown and described.

Within the claims, reference to an element in the singular is not intended to mean “one and only one” unless specifically stated as such, but rather as “one or more” or “at least one”. Unless specifically stated otherwise, the term “some” refers to one or more. No claim element is to be construed under the provision of 35 U.S.C. § 112(f) unless the element is expressly recited using the phrase “means for” or “step for”. All structural and functional equivalents to the elements of the various embodiments described in the present disclosure that are known or come later to be known to those of ordinary skill in the relevant art are expressly incorporated herein by reference and are intended to be encompassed by the claims. Moreover, nothing disclosed in the present disclosure is intended to be dedicated to the public regardless of whether such disclosure is explicitly recited in the claims.

We claim:

1. A dilator for insertion within a catheter, comprising:
a body having an external surface extending from a proximal end to a distal end and having a first portion, a second portion, and a third portion;
wherein:
the second portion is disposed between the first portion and the third portion and has a consistent outer diameter to the first portion and the third portion;
the external surface in the first portion includes a coating with a first coefficient of friction;
the external surface in the second portion exhibits a second coefficient of friction greater than the first coefficient of friction, and
the external surface in the third portion exhibits a third coefficient of friction less than the second coefficient of friction.

2. The dilator of claim 1, wherein the body comprises a thermoplastic elastomer which includes a blend of polyether polymer and polyamide polymer.

3. The dilator of claim 1, wherein the first portion and the third portion are coated with a hydrophilic coating that is not applied to the second portion.

4. The dilator of claim 1, wherein the first portion and the third portion are coated with a lubricious coating that is not applied to the second portion.

5. The dilator of claim 1, wherein the distal end has at least a partially rounded, bulb-shaped end having an outer diameter greater than an outer diameter of any of the first portion, the second portion, and the third portion.

6. The dilator of claim 1, wherein a lumen is defined in the body that extends axially from a proximal opening at the proximal end, through the body to a distal opening at the distal end.

7. The dilator of claim 1, wherein a lumen is defined in the body that extends axially from a proximal opening at the proximal end, through the body towards the distal end.

8. The dilator of claim 1, wherein a lumen is defined in the body that extends axially from a proximal opening at the proximal end, wherein the lumen includes at least one delivery hole defined in the first portion.

9. The dilator of claim 8, further comprising a guidewire disposed within the lumen.

10. The dilator of claim 1, wherein the distal end includes an atraumatic tip.

11. The dilator of claim 1, wherein a plasma treatment imparted to the second portion defines the second coefficient of friction to be higher than the first coefficient of friction and the third coefficient of friction.

12. The dilator of claim 1, wherein the body further comprises a fourth portion discontinuous from the second portion such that the third portion is disposed between the second portion and the fourth portion, wherein the external surface in the fourth portion exhibits the second coefficient of friction.

13. The dilator of claim 1, wherein the first coefficient of friction is substantially equal to the third coefficient of friction.

14. A method, comprising:
selecting a dilator for insertion into a catheter;
gripping the dilator via a gripping surface having a higher coefficient of friction than an average coefficient of friction along a length of the dilator;
inserting the dilator through the catheter; and
in response to reaching a target location in a biological subject accessible via the catheter, withdrawing the dilator from the catheter.

15. The method of claim 14, further comprising, in response to inserting at least a portion of the gripping surface into the catheter, repositioning where the dilator is gripped to a second gripping surface that is located further from a tip of the dilator than the gripping surface.

16. The method of claim 14, further comprising, in response to withdrawing at least a portion of the gripping surface from the catheter, repositioning where the dilator is gripped to a second gripping surface that is located closer to a tip of the dilator than the gripping surface.

17. The method of claim 14, further comprising, in response to reaching the target location, delivering a contrast agent or a therapeutic agent to the target location via delivery holes and a lumen formed in the dilator that are in fluid communication with the target location.

18. A method, comprising:
receiving a first section and a second section of a base material for construction of a dilator;
melding the first section to the second section to form the dilator;
covering some, but not all of, the dilator with a sleeve to produce a covered portion and an exposed portion;
applying a coating to the dilator; and
removing the sleeve from the dilator to yield a first portion, corresponding to a first one of the covered portion and the exposed portion, and a second portion, corresponding to a second one of the covered portion and the exposed portion, wherein the first portion and the second portion exhibit different coefficients of friction.

19. The method of claim 18, further comprising:
forming, via laser drilling, delivery holes from an external surface of the dilator to a lumen defined axially within the dilator.

20. The method of claim 18, wherein the coating is applied to the covered portion at a lower concentration than to the exposed portion.
